(19) United States
(12) Patent Application Publication
(10) Pub. No.: US 2021/0047646 A1
(43) Pub. Date: Feb. 18, 2021

(54) Compositions Comprising Immune System Activators and Method of Using Same

(71) Applicant: The Trustees of Princeton University, Princeton, NJ (US)

(72) Inventors: Alisha Chitrakar, Princeton, NJ (US); Alexei Korennykh, Princeton, NJ (US)

(21) Appl. No.: 17/044,195

(22) PCT Filed: Apr. 9, 2019
(86) PCT No.: PCT/US19/26446

§ 371 Date: Sep. 30, 2020

Related U.S. Application Data
(60) Provisional application No. 62/654,923, filed on Apr. 9, 2018.
(60) Provisional application No. 62/803,058, filed on Feb. 8, 2019.

(51) Int. Cl. C12N 15/113; A61K 31/7125; A61K 45/06

(52) U.S. Cl. CPC C12N 15/1137; A61K 31/7125; C12N 2310/321; C12N 2310/11; C12N 2310/315; A61K 45/06

(57) ABSTRACT

The present invention relates, in various embodiments, to compositions comprising synthetic DNA molecules, and methods of using such compositions to enhance an immune response to cancer, for example, by activating the RNase L pathway in cells without inducing immunosuppressive effects caused by other agents that are known to activate this pathway.

### RELATED APPLICATIONS

This application claims the benefit of U.S. Provisional Application No. 62/803,058, filed Feb. 8, 2019 and U.S. Provisional Application No. 62/654,923, filed on Apr. 9, 2018. The entire teachings of the above applications are incorporated herein by reference.

### GOVERNMENT SUPPORT

This invention was made with government support under Grant No. GM110161 awarded by the National Institutes of Health. The government has certain rights in the invention.

### BACKGROUND

The Interferon (IFN) inducible 2′-5′-oligoadenylate synthetase (OAS)/endoribonuclease RNase L pathway plays an important role in innate immunity against both pathogenic viral infections and tumor cells through cleavage of viral and cellular single-stranded RNA (Silverman, R. H., “Viral Encounters with 2′,5′-Oligoadenylate Synthetase and RNase L during the interferon Antiviral Response,” J. Virol., 81(23) 12720-12729 (2007); Choi U. Y., et al., “Oligoadenylate synthase-like (OASL) proteins: dual functions and associations with diseases,” Experimental Molecular Medicine, 47: e144 (2015)). IFN signaling induces transcription of OAS genes through IFN-stimulated response elements in OAS gene promoters and the OAS enzyme further generates dsRNA molecules which activate RNase L. However, the use of Interferons as therapeutic agents for the treatment of cancer and other diseases has potential drawbacks, as IFN signaling can have immunosuppressive effects (Minn., A. J., et al., “Interferons and the Immunogenic Effects of Cancer Therapy,” Trends Immunol., 36(11): 725-737 (2015)). Thus, there is a need for alternative strategies for inducing RNase L that avoid the immunosuppressive effects of IFN.

### SUMMARY

The present invention generally relates to compositions and methods for activating the RNase L enzyme in vivo (e.g., in a cell, in a subject).

Accordingly, in some embodiments, the invention relates to a composition comprising a DNA oligonucleotide molecule, wherein the DNA oligonucleotide molecule comprises: a) a phosphorothioate linkage; and b) a 2′-O-methyl RNA base at the 5′ end, the 3′ end or both the 5′ and 3′ ends of the DNA oligonucleotide molecule. In particular embodiments, the DNA oligonucleotide molecule is a single stranded DNA (ssDNA) molecule.

In additional embodiments, the invention as described herein relates to a pharmaceutical composition comprising a DNA oligonucleotide molecule and a pharmaceutically acceptable carrier.

In further embodiments, the invention relates to a method for treating cancer in a subject in need thereof, comprising the step of administering to the subject an effective amount of a DNA oligonucleotide molecule, wherein the DNA oligonucleotide molecule comprises a phosphorothioate linkage, and wherein the DNA oligonucleotide molecule comprises at least one strand of more than 10 contiguous deoxyribonucleotide bases.

In certain embodiments, the invention relates to a method for activating an RNase L enzyme in a cell, comprising the step of contacting the cell with a DNA oligonucleotide molecule, wherein the DNA oligonucleotide molecule comprises a phosphorothioate linkage, and wherein the DNA oligonucleotide molecule comprises at least one strand of more than 10 contiguous deoxyribonucleotide bases.

The compositions and methods described herein are useful, inter alia, for activating the RNase L enzyme independent of the IFN pathway, thereby avoiding the immunosuppressive effects of IFNs.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A-1E. Activation of RNase L by DNA oligonucleotide with phosphorothioate modification and 2′ O Methyl RNA bases. A) northern blot in left panel depicting RNase L mediated ribososmal RNA (rRNA) cleavage on a Bioanalyzer rRNA, in wild type A549 cells transfected with Antisense oligonucleotide targeting Dicer, with either phosphorothioate modification (Dicer 1) or 2′ O Methyl RNA bases (Dicer 2) show or both (Dicer). A negative control sequence with the same modifications targeting a non-essential gene GGA2 also activates RNase L. North blot in right panel shows in RNase L knock out A549 cells transfected with the same oligonucleotides as in left panel, there is no rRNA cleavage. B) Northern Blot showing activation of RNase L in A549 cells by phosphorothioate containing oligonucleotide with a unique sequence with phosphorothioate modification, that does not target any mRNA (Non-targeted) and a Randomer sequence with phosphorothioate modification and 2′ O-methyl RNA bases, wherein the randomer sequence is a stronger activator of RNase L. C) Northern Blot showing RNase L mediated rRNA cleavage fragments in A549 cells transfected with oligonucleotide with phosphorothioate modification and 2′ O Methyl RNA bases (GGA2) appear at 9 hrs post. D) Rtcb-ligation quantitative PCR showing higher level of formation of Histidine-transfer RNA (tRNA-His) and 28S-4032 cleavage fragments (as depicted by increase in fragments compared to control ASO), in RNase L wild type (RNK WT) as compared to RNase L knock out (RNL KO) cells and E) Northern Blot showing RNase L mediated tRNA-His cleavage products in A549 cells transfected with oligonucleotide with phosphorothioate modification and 2′ O Methyl RNA bases (GGA2) appear at 9 and 12 hrs.

FIG. 2A-2B. Effect of Tag orientation and oligonucleotide size on RNase L activation. A) northern blot showing oligonucleotides sequence with phosphorothioate modification and 2′ O-methyl RNA bases, comprising 5′ Biotin tag, weakens RNase L activation. B) Northern blot oligonucleotides sequence with phosphorothioate modification and 2′ O-methyl RNA bases that are 10 and 5 nucleotide bases long cannot activate RNase L.

FIG. 3. Dicer independent activation of RNase L. Western blot showing that treatment with antisense oligonucleotide targeting Dicer comprising phosphorothioate modification or 2′ O-methyl RNA bases or both, do not reduce Dicer protein levels, even though RNase L activation can be detected as shown in FIG. 1A. GAPDH levels were used as a control.

FIG. 4. Phosphorothioate DNA oligonucleotide causes PARP cleavage in A549 human adenocarcinoma cells. Cleavage of PARP is a marker of cells undergoing apoptosis. Western blot analysis against PARP shows that DNA oligonucleotide containing phosphorothioate modification results in cleavage of full length PARP. Dicer-1 sequence but not Dicer-2 causes PARP cleavage which suggests the phosphorothioate modifications are necessary.

FIGS. 5A-5E. Phosphorothioate oligonucleotide stress induces double stranded RNA. FIG. 5A) (Top) Schematic for transcription inhibition experiment with Actinomycin D. (Bottom) Bioanalyzer analysis of rRNA cleavage with 1 μg/mL Actinomycin D and 2 ng/mL Poly IC or 50 nM of the modified oligonucleotide GGA2. FIG. 5B) Bioanalyzer analysis of rRNA cleavage in WT and OAS KO A549 cells treated with 50 nM of the indicated modified oligonucleotides. FIG. 5C) GSEA profiling of RNAseq data. (Top) Exon counts (Bottom) Intron counts for ASO induced reads FIG. 5D) qPCR of interferon stimulated genes on cells treated with 1 μg/mL Poly IC for 4 hrs and/or 50 nM of Randomer oligonucleotide for 12 hours. FIG. 5E) Schematic for ASO induced transcriptional dsRNA response.

### DETAILED DESCRIPTION

Mammalian cells activate RNase L to combat stress resulting from build-up of double stranded RNA (dsRNA) (Gantier, M. P. and Williams, B. R. G., “The response of mammalian cells to double-stranded RNA,” Cytokine Growth Factor Rev. 18(5-6): 363-371 (2007)). The antiviral, anti-proliferative and immunomodulatory activities of RNase L make it a potential therapeutic target in the treatment of disease (Silverman, R. H., “Implications for RNase L in prostate cancer biology,” Biochemistry, 25; 42(7):1805-12 (2003), and Meyer, M. S., et al., “Genetic variation in RNASEL associated with prostate cancer risk and progression,” Carcinogenesis, 31(9): 1597-1603 (2010)).

The present invention is based, in part, on the discovery that specifically modified DNA oligonucleotides can activate the RNase L enzyme in a cell.

A description of example embodiments follows.

Compositions Comprising Oligonucleotide Molecules that Activate RNase L Enzyme

The present invention relates, in various embodiments, to a composition comprising an oligonucleotide molecule (e.g., a DNA oligonucleotide molecule, a plurality of DNA oligonucleotide molecules), wherein the oligonucleotide molecule comprises: a) a phosphorothioate linkage; and b) a 2′-O-methyl RNA base at the 5′ end, the 3′ end or both the 5′ and 3′ ends of the oligonucleotide molecule.

As used herein, the term “oligonucleotide” refers to a nucleic acid polymer having about 10 to about 100 nucleotide monomers. An oligonucleotide can be single- or double-stranded, and can be DNA (e.g., cDNA), RNA, or hybrid polymers (e.g., DNA/RNA). An oligonucleotide can be unmodified or modified, and/or can contain natural and/or non-natural or derivatized nucleotide bases. Oligonucleotides can also include, for example, conformationally restricted nucleic acids (e.g., “locked nucleic acids” or “LNAs,” such as described in Nielsen et al., J. Biomol. Struct. Dyn. 17:175-91, 1999), morpholinos, glycol nucleic acids (GNA) and threose nucleic acids (TNA).

In certain embodiments, the oligonucleotide molecules in the compositions described herein are DNA oligonucleotide molecules. In some embodiments, the DNA oligonucleotide molecules in the compositions described herein are single stranded DNA (ssDNA) molecules. In other embodiments, the DNA oligonucleotide molecules in the compositions described herein are double stranded DNA (dsDNA) molecules.

Examples of nucleotide bases that can be incorporated into oligonucleotide molecules useful in the present invention include adenosine (A), thymidine (T), guanidine (G), cytidine (C), uridine (U), 5-methylcytosine, 5-(Hydroxymethyl)cytosine, 5-Formylcytosine, 5-Carboxycytosine, (a/b-D-Glucosyl)-5-(hydroxymethylcytosine, N4-Methylcytosine, 1-a-D-Glc/Gal-5-hydroxycytosine, a-Putrescinylthymine, 2′-Deoxypseudouridine, 2′-Deoxyuridine, 2-Thiothymidine, 4-Thio-2′-deoxyuridine, 4-Thiothymidine, 5′ Aminothymidine, 5-(1-Pyrenylethynyl)-2′-deoxyuridine, 5-(C2-EDTA)-2′-deoxyuridine, 5-(Carboxy)vinyl-2′-deoxyuridine, 5,6-Dihydro-2′-deoxyuridine, 5-Bromo-2′-deoxycytidine, 5-Bromo-2′-deoxyuridine, 5-Carboxy-2′-deoxycytidine, 5-Fluoro-2′-deoxyuridine, 5-Formyl-2′-deoxycytidine, 5-Hydroxy-2′-deoxycytidine, 5-Hydroxy-2′-deoxyuridine, 5-Hydroxymethyl-2′-deoxycytidine, 5-Hydroxymethyl-2′-deoxyuridine, 5-Iodo-2′-deoxycytidine, 5-Iodo-2′-deoxyuridine, 5-Methyl-2′-deoxycytidine, 5-Methyl-2′-deoxyisocytidine, 5-Propynyl-2′-deoxycytidine, 5-Propynyl-2′-deoxyuridine, 6-O-(TMP)-5-F-2′-deoxyuridine, C4-(1,2,4-Triazol-1-yl)-2′-deoxyuridine, C8-Alkyne-thymidine, dT-Ferrocene, N4-Ethyl-2′-deoxycytidine, O4-Methylthymidine, Pyrrolo-2′-deoxycytidine, Thymidine Glyco, 2,6-Diaminopurine-2′-deoxyriboside, 2-Aminopurine-2′-deoxyriboside, 6-Thio-2′-deoxyguanosine, 7-Deaza-2′-deoxyadenosine, 7-Deaza-2′-deoxyguanosine, 7-Deaza-2′-deoxyxanthosine, 7-Deaza-8-aza-2′-deoxyadenosine, 8-5′(5′S)-Cyclo-2′-deoxyadenosine, 8-Amino-2′-deoxyadenosine, 8-Amino-2′-deoxyguanosine, 8-Bromo-2′-deoxyadenosine, 8-Bromo-2′-deoxyguanosine, 8-Deuterated-2′-deoxyguanosine, 8-Oxo-2′-deoxyadenosine, 8-Oxo-2′-deoxyguanosine, Etheno-2′-deoxyadenosine, Formylindole, N⁶-Methyl-2′-deoxyadenosine, O⁶-Methyl-2′-deoxyguanosine and OX⁶-Phenyl-2′deoxyinosine.

The oligonucleotides (e.g., DNA oligonucleotides) in the compositions described herein contain at least one phosphorothioate linkage between adjacent nucleotides. As used herein, the term “phosphorothioate linkage” refers to an internucleotide linkage involving a phosphorothioate (PS) bond that substitutes a sulfur atom for a non-bridging oxygen in the phosphate backbone of an nucleic acid as illustrated in Structure below.

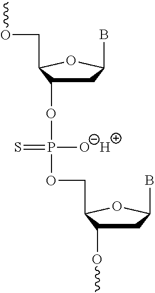

In some embodiments, the DNA oligonucleotide molecule comprises a phosphorothioate linkage between each deoxyribonucleotide base in at least one of the strands of the DNA oligonucleotide molecule, such that all contiguous nucleotides in the strand are linked by phosphorothioate linkages. In particular embodiments, the DNA oligonucleotide molecule has phosphorothioate linkages only at the 5′ end and the 3′ end of the DNA oligonucleotide molecule. In certain embodiments, the DNA oligonucleotide molecule has phosphorothioate linkages either at the 5′ end or the 3′ end of the DNA oligonucleotide molecule.

In some embodiments, the DNA oligonucleotide molecule comprises only one phosphorothioate linkage. In other embodiments, the DNA oligonucleotide molecule comprises at least two (e.g., 2, 3, 4, 5, 6, etc.) phosphorothioate linkages. In some embodiments, the DNA oligonucleotide molecule comprises phosphorothioate linkages in less than half of the total linkages between the deoxyribonucleotide bases in the DNA oligonucleotide molecule. In further embodiments, the DNA oligonucleotide molecule comprises phosphorothioate linkages in about half of the total linkages between the deoxyribonucleotide bases in the DNA oligonucleotide molecule. In particular embodiments, the DNA oligonucleotide molecule comprises phosphorothioate linkages in more than half of the total linkages between the deoxyribonucleotide bases in the DNA oligonucleotide molecule.

The oligonucleotides of the invention can be produced recombinantly or synthetically, using routine methods and reagents that are well known in the art and available commercially. For example, DNA oligonucleotide molecules containing one or more phosphorothioate linkages can be produced by incorporating a modified, phosphorothioate deoxyribonucleotide base into a growing polynucleotide chain. The oligonucleotides of the invention are also available commercially, for example, at Integrated DNA Technology.

The oligonucleotides (e.g., DNA oligonucleotides) in the compositions described herein can also contain, in various embodiments, a 2′-O-methyl RNA base. The 2′-O-methyl RNA base can be at the 5′ end, the 3′ end or both the 5′ and 3′ ends of the oligonucleotide molecule.

As used herein, the term “2′-O-methyl RNA base” refers to modification of ribonucleotide, where a methyl group is added to the 2′ hydroxyl of the ribose moiety of the nucleoside, producing a methoxy group. In various embodiments, the composition as described herein comprises DNA oligonucleotide molecules that are modified by addition of one or more 2′-O-methyl RNA bases, for example, at either the 3′ end, the 5′ end, or both the 3′ and the 5′ ends.

In some embodiments, the DNA oligonucleotide molecule comprises at least one strand of 10 or more contiguous deoxyribonucleotide bases. In other embodiments, the DNA oligonucleotide molecule comprises more than 10 nucleotide bases (e.g., 11, 12, 13, 14, 15, etc., nucleotide bases). In particular embodiments, the DNA oligonucleotide molecule comprises up to 30 nucleotide bases (e.g., 20, 25, 30, etc. nucleotide bases). In further embodiments, the DNA oligonucleotide molecule comprises more than 30 nucleotide bases. In certain embodiments, the DNA oligonucleotide molecule comprises about 23 nucleotide bases.

In some embodiments, the DNA oligonucleotide molecule comprises a nucleotide sequence that is not identical to a mammalian genomic nucleotide sequence having the same length. In particular embodiments, the DNA oligonucleotide molecule comprises a nucleotide sequence that has less than 50% identity to a mammalian genomic nucleotide sequence of the same length.

In some embodiments, a composition of the present invention comprises a plurality of DNA oligonucleotide molecules described herein. In certain embodiments, each DNA oligonucleotide molecule comprises a different sequence of deoxyribonucleotide bases relative to the other DNA oligonucleotides in the composition. The DNA oligonucleotide molecules within the plurality can be of the same length or have different lengths, or can be a mixture thereof.

In particular embodiments, the DNA oligonucleotide molecules in the compositions described herein comprise, consists essentially of, or consist of, a sequence shown in Table 1 herein.

TABLE1
Sequences of the antisense oligonucleotides.

| Antisense Oligos | Sequence |
|---|---|
| Ctrl | GCCAGATATACGCGTTGAC |
| (SEQ ID NO: 3) | |
| Dicer | mG*mC*mU*mG*mA*d(C*C*T*T*T*T*T* |
| (SEQ ID NO: 4) | G*C*T)*mU*mC*mU*mC*mA |
| GGA2 | mC*mA*mU*mC*mU*d(C*C*A*G*C*A*C* |
| (SEQ ID NO: 5) | C*G*T*T*A*A)*mG*mG*mC*mA*mU |
| Dicer-1 | d(G*C*T*G*A*C*C*T*T*T*T*T*G*C*T |
| (SEQ ID NO. 6) | *T*C*T*C*A) |
| Dicer-2 | mGmCmUmGmAd(CCTTTTTGCT)mUmCmUmC |
| (SEQ ID NO. 7) | mA |
| Non-targeted | d(A*C*A*C*T*C*T*T*T*C*C*C*T*A*C |
| (SEQ ID NO. 8) | *A*C*G*A*C*G*C*T*C*T*T*C*C*G*A* |
| | T*C) |
| Randomer | mN*mN*mN*mN*mN*d(N*N*N*N*N*N*N* |
| (SEQ ID NO. 9) | N*N*N*N*N*N)*mN*mN*mN*mN*mN |
| 10mer | d(N*N*N*N*N*N*N*N*N*N) |
| (SEQ ID NO. 10) | |
| 5 mer | d(N*N*N*N*N) |
| (SEQ ID NO. 11) | |

In some embodiments, the compositions described herein can activate the RNase L enzyme in a cell without activating the interferon pathway or inducing interferons in a cell. In particular embodiments, the compositions can activate the RNase L enzyme in a cell without affecting Dicer activity or expression.

In some embodiments, the compositions described herein comprise a concentration of DNA oligonucleotide molecules of at least about 50 nM. In certain embodiments, the compositions comprise a concentration of DNA oligonucleotide molecules of at least about 100 nM. In particular embodiments, the compositions comprise a concentration of DNA oligonucleotide molecules of at least about 300 nM.

In some embodiments, the compositions comprise a concentration of DNA oligonucleotide molecules that can induce cell death/apoptosis in a cell. In certain embodiments, the compositions comprise a concentration of DNA oligonucleotide molecules that can inhibit protein synthesis in a cell. In particular embodiments, the compositions comprise a concentration of DNA oligonucleotide molecules that can inhibit proliferation of a cell.

In some embodiments, the compositions described herein are useful for killing virus-infected cells.

In certain embodiments, the compositions described herein are useful for activating (e.g., initiating, maintaining and/or enhancing) an immune response in a subject (e.g., a patient). Examples of immune responses that can be activated using the methods and compositions described herein include, but are not limited to, an RNase L pathway, a T cell response, a macrophage response, an NK cell response, a dendritic cell response, a neutrophil response and a B cell response. In a particular embodiment, the immune response is an immune response to a tumor or tumor antigen, also referred to herein as an “anti-tumor immune response”. An anti-tumor response can be directed to, for example, tumor control, (e.g., delaying and/or halting tumor growth and/or metastasis), tumor killing (e.g., causing the death of cancerous cells in a tumor), or both.

The compositions described herein can be formulated for administration to a subject (e.g., a human). Accordingly, in various embodiments, the compositions described herein further comprise one or more pharmaceutically acceptable carriers or excipients. Suitable pharmaceutical carriers typically will contain inert ingredients that do not interact with the agent or nucleic acid. Examples of pharmaceutical carriers include, for example, sterile water, physiological saline, bacteriostatic saline (saline containing about 0.9% mg/ml benzyl alcohol), phosphate-buffered saline, Hank's solution, Ringer's lactate, solutions appropriate for supporting the health of immune cells (e.g., solutions containing glucose, amino acids, growth factors, and/or other nutrients or immune stimulators), and the like. Formulations can also include small amounts of substances that enhance the effectiveness of the active ingredient (e.g., emulsifying agents, solubilizing agents, pH buffering agents, wetting agents). For inhalation, the agent can be solubilized and loaded into a suitable dispenser for administration (e.g., an atomizer or nebulizer or pressurized aerosol dispenser).

Standard pharmaceutical formulation techniques can be employed, such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's lactate and the like. Formulations can also include small amounts of substances that enhance the effectiveness of the active ingredient (e.g., emulsifying, solubilizing, pH buffering, wetting agents). Methods of encapsulation compositions (such as in a coating of hard gelatin or cyclodextran) are known in the art. For inhalation, the agent can be solubilized and loaded into a suitable dispenser for administration (e.g., an atomizer or nebulizer or pressurized aerosol dispenser).

An oligonucleotide molecule in the compositions described herein can be administered to a subject as a neutral compound or as a salt or ester. Pharmaceutically acceptable salts include those formed with free amino groups such as those derived from hydrochloric, phosphoric, acetic, oxalic or tartaric acids, and those formed with free carboxyl groups such as those derived from sodium, potassium, ammonium, calcium, ferric hydroxides, isopropylamine, triethylamine, 2-ethylamino ethanol, histidine, procaine, etc. Salts of compounds containing an amine or other basic group can be obtained, for example, by reacting with a suitable organic or inorganic acid, such as hydrogen chloride, hydrogen bromide, acetic acid, perchloric acid and the like. Compounds with a quaternary ammonium group also contain a counteranion such as chloride, bromide, iodide, acetate, perchlorate and the like. Salts of compounds containing a carboxylic acid or other acidic functional group can be prepared by reacting with a suitable base, for example, a hydroxide base. Salts of acidic functional groups contain a countercation such as sodium or potassium.

In other embodiments, the pharmaceutically acceptable carrier is selected from a liposome, a nanoparticle, an exosome, a micelle, a polymeric matrix or a gel matrix, wherein the DNA oligonucleotide molecule is contained in, or is in a complex with, the liposome, nanoparticle, exosome, micelle, polymeric matrix or gel matrix.

In some embodiments, the compositions described herein include one or more additional therapeutic agents (e.g., a chemotherapeutic agent and/or an immunomodulatory agent). In some embodiments, the compositions described herein comprise at least one chemotherapeutic drug. Examples of chemotherapeutic drugs include a radionuclide, an immunomodulator, a hormone, a hormone antagonist, an enzyme, an anti-sense oligonucleotide, an siRNA, an enzyme inhibitor, a photoactive therapeutic agent, a cytotoxic agent, a drug, a toxin, an angiogenesis inhibitor and a pro-apoptotic agent. In certain other embodiments, the composition comprises at least one chemotherapeutic drug selected from the group consisting of is selected from the group consisting of nitrogen mustards, ethylenimine derivatives, alkyl sulfonates, nitrosoureas, gemcitabine, triazenes, folic acid analogs, anthracyclines, taxanes, COX-2 inhibitors, pyrimidine analogs, purine analogs, antibiotics, enzyme inhibitors, epipodophyllotoxins, platinum coordination complexes, vinca alkaloids, substituted ureas, methyl hydrazine derivatives, adrenocortical suppressants, hormone antagonists, endostatin, taxols, camptothecins, SN-38, doxorubicins and their analogs, antimetabolites, alkylating agents, antimitotics, anti-angiogenic agents, tyrosine kinase inhibitors, mTOR inhibitors, heat shock protein (HSP90) inhibitors, proteosome inhibitors, HDAC inhibitors, pro-apoptotic agents, methotrexate and CPT-11.

In some embodiments, the compositions described herein comprise at least one immunomodulatory agent. Examples of immunomodulatory agents include a cytokine, a stem cell growth factor, a lymphotoxin, a hematopoietic factor, a colony stimulating factor (CSF), an interleukin (IL), an interferon (IFN), a stem cell growth factor, erythropoietin, thrombopoietin, tumor necrosis factor (TNF), granulocyte-colony stimulating factor (G-CSF), granulocyte macrophage-colony stimulating factor (GM-CSF), interferon-α, interferon-β, interferon-γ, antibodies against immune checkpoints and the stem cell growth factor designated “S1 factor”. Examples of cytokines include human growth hormone, N-methionyl human growth hormone, bovine growth hormone, parathyroid hormone, thyroxine, insulin, proinsulin, relaxin, prorelaxin, glycoprotein follicle stimulating hormone (FSH), thyroid stimulating hormone (TSH), luteinizing hormone (LH), placenta growth factor (PlGF), hepatic growth factor, prostaglandin, fibroblast growth factor, prolactin, placental lactogen, OB protein, tumor necrosis factor-α, tumor necrosis factor-β, mullerian-inhibiting substance, mouse gonadotropin-associated peptide, inhibin, activin, vascular endothelial growth factor, integrin, thrombopoietin (TPO), NGF-β, platelet-growth factor, TGF-α, TGF-β, insulin-like growth factor-I, insulin-like growth factor-II, erythropoietin (EPO), osteoinductive factors, interferon-α, interferon-β, interferon-γ, macrophage-CSF (M-CSF), IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, TNF-α and LT. Examples of antibodies against immune checkpoints include antibodies against CTLA4, PD1, PD2, PDL-1, PDL-2, B7-1, B7-1, LAG-3, TIM-3, KIRs, 4-IBB, 4-IBBL, TIGIT, Galectin-9, GITR, GITRL, DR3, HVEM, TL1A, CD27, CD28, CD30, CD40, CD40L, CD80, CD86, CD96, Nectin, OX-40, OX-40L, ICOS CD155, CD226, CD258, CD272, and CD276.

In some embodiments, the compositions described herein comprise at least one anti-viral drug. Examples of anti-viral drugs include Abacavir, Acyclovir (Aciclovir), Adefovir, Amantadine, Amprenavir, Ampligen, Arbidol, Atazanavir, Atripla, Balavir, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fixed dose combination (antiretroviral), Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, NexavirNitazoxanide, Nucleoside analogues, Norvir, Oseltamivir (Tamiflu), Peginterferon alfa-2a, Penciclovir, Peramivir, Pleconaril, Podophyllotoxin, Protease inhibitor (pharmacology), Raltegravir, Reverse transcriptase inhibitor, Ribavirin, Rimantadine, Ritonavir, Pyramidine, Saquinavir, Sofosbuvir, Stavudine, Synergistic enhancer (antiretroviral), Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza) and Zidovudine.

### Methods of Treating a Subject Methods of Activating an RNase L Enzyme in a Cell

The present invention also relates, in certain embodiments, to a method for treating a subject in need thereof (e.g., a subject having cancer), comprising the step of administering to the subject an effective amount of an oligonucleotide molecule (e.g., a DNA oligonucleotide molecule) that comprises at least one phosphorothioate linkage. In some embodiments, the DNA oligonucleotide molecule comprises at least one strand of 10 or more contiguous deoxyribonucleotide bases.

In certain embodiments, the DNA oligonucleotide molecule also comprises a 2′-O-methyl RNA base. The 2′-O-methyl RNA base can be at the 5′ end, the 3′ end or both the 5′ and 3′ ends of the DNA oligonucleotide molecule.

In some embodiments, the method described here in can be used for treating cancer in a subject, for example, by killing cancer cells in a subject, by inhibiting proliferation of cancer cells in a subject, by activating an immune response to a cancer in a subject, and/or by activating RNase L pathway in cancer cells in a subject.

As used herein, “subject” refers to a mammal (e.g., human, non-human primate, cow, sheep, goat, horse, dog, cat, rabbis, guinea pig, rat, mouse). In some embodiments, the subject is a human. A “subject in need thereof” refers to a subject (e.g., patient) who has, or is at risk for developing, a disease or condition (e.g., cancer or a viral infection) that can be treated (e.g., improved, ameliorated, prevented) by administration of a composition described herein.

As used herein, the terms “treat,” “treating,” or “treatment,” mean to counteract a medical condition (e.g., a condition related to cancer, viral infection) to the extent that the medical condition is improved according to a clinically-acceptable standard (e.g., reduction in tumor formation, size, growth or metastasis).

In some embodiments, the subject in need thereof has cancer. The cancer can be a solid tumor, a leukemia, a lymphoma or a myeloma. In particular embodiments, the subject in need thereof has a solid tumor, such as a breast tumor, a colon tumor, a lung tumor, a pancreatic tumor, a prostate tumor, a bone tumor, a skin tumor (e.g., melanoma, squamous cell carcinoma), a brain tumor, a head and neck tumor, a lymphoid tumor, or a liver tumor.

As used herein, an “effective amount” refers to an amount of a composition or therapeutic agent as described herein that, when administered to a subject, is sufficient to achieve a desired therapeutic effect in the subject under the conditions of administration, such as an amount sufficient to promote (e.g., initiate, maintain and/or enhance) an immune response (e.g., an RNase L response) to a tumor in the subject.

The therapeutic effectiveness of an oligonucleotide molecule or composition described herein can be determined by any suitable method known to those of skill in the art (e.g., in situ immunohistochemistry, imaging (ultrasound, CT scan, MRI, NMR), ³H-thymidine incorporation) using any suitable standard (e.g., inhibition of tumor formation, tumor growth (proliferation, size), tumor vascularization, tumor progression (invasion, metastasis) and/or chemoresistance).

In certain embodiments, the method described herein can be used in combination with administration of at least one chemotherapeutic drug/agent. In further embodiments, the method described herein can be used in combination with administration of at least one immunomodulatory drug/agent. In certain embodiments, the method described herein can be used in combination with administration of at least one anti-viral drug/agent.

When administered in a combination therapy, administration of an oligonucleotide molecule or composition described herein can be done before, after or concurrently with the other therapeutic agent (e.g., administration of a chemotherapeutic agent, such a paclitaxel or doxorubicin). When co-administered simultaneously (e.g., concurrently), the oligonucleotide molecule or composition and other therapeutic agent can be in separate formulations or the same formulation. Alternatively, the oligonucleotide molecule or composition and other therapy can be administered sequentially, as separate compositions, within an appropriate time frame (e.g., a cancer treatment session/interval such as 1.5 to 5 hours) as determined by a skilled clinician (e.g., a time sufficient to allow an overlap of the pharmaceutical effects of the therapies).

The compositions described herein can be administered to a subject in need thereof by a variety of routes of administration, including, for example, oral (e.g., dietary, in the form of a nutritional supplement), topical, transdermal, rectal, parenteral (e.g., intra-arterial, intravenous, intramuscular, subcutaneous, intradermal), intravenous infusion, and inhalation (e.g., intrabronchial, intranasal or oral inhalation, intranasal drops), depending on the agent and the particular disease (e.g., cancer) to be treated.

Administration can be local or systemic, as indicated. The chosen mode of administration can vary depending on the particular agent selected. The actual dose of a therapeutic agent and treatment regimen can be determined by a skilled physician, taking into account the nature of the condition being treated, and patient characteristics.

In various embodiments, the invention further relates to a method of activating a RNase L enzyme (e.g., NCBI Reference Sequence: NP_066956.1/UniProtKB/Swiss-Prot: Q05823.1; or UniProtKB/Swiss-Prot: Q05823.2) in a cell, by contacting the cell with any of the DNA oligonucleotide molecules comprising a phosphorothioate linkage described herein, or any composition described herein comprising such DNA oligonucleotide molecules.

The RNase L enzyme activated by the methods and compositions described herein can be, for example, a canonical or wild-type RNase L enzyme (e.g., SEQ. ID. NO 1 or SEQ. ID. NO 2), or naturally occurring variants thereof. A variant of a RNase L enzyme variant can have an amino acid sequence that is at least 50% identical, for example, about 50%, 60%, 70%, 80%, 90%, 95%, 98% or 99% identical, to SEQ ID. NO.: 1 or SEQ ID. NO.: 2.

SEQ ID NO: 1
Human 25-5′A dependent endoribonuclease/RNase
Lisoform 1 (741 amino acids)
MESRDHNNPQEGPTSSSGRRAAVEDNHLLIKAVQNEDVDLVQQLLEGGAN
VNFQEEEGGWTPLHNAVQMSREDIVELLLRHGADPVLRKKNGATPFILAA
IAGSVKLLKLFLSKGADVNECDFYGFTAFMEAAVYGKVKALKFLYKRGAN
VNLRRKTKEDQERLRKGGATALMDAAEKGHVEVLKILLDEMGADVNACDN
MGRNALIHALLSSDDSDVEAITHLLLDHGADVNVRGERGKTPLILAVEKK
HLGLVQRLLEQEHIEINDTDSDGKTALLLAVELKLKKIAELLCKRGASTD
CGDLVMTARRNYDHSLVKVLLSHGAKEDFHPPAEDWKPQSSHWGAALKDL
HRIYRPMIGKLKFFIDEKYKIADTSEGGIYLGFYEKQEVAVKTFCEGSPR
AQREVSCLQSSRENSHLVTFYGSESHRGHLFVCVTLCEQTLEACLDVHRG
EDVENEEDEFARNVLSSIFKAVQELHLSCGYTHQDLQPQNILIDSKKAAH
LADFDKSIKWAGDPQEVKRDLEDLGRLVLYVVKKGSISFEDLKAQSNEEV
VQLSPDEETKDLIHRLFHPGEHVRDCLSDLLGHPFFWTWESRYRTLRNVG
NESDIKTRKSESEILRLLQPGPSEHSKSFDKWTTKINECVMKKMNKFYEK
RGNFYQNTVGDLLKFIRNLGEHIDEEKHKKMKLKIGDPSLYFQKTFPDLV
IYVYTKLQNTEYRKHFPQTHSPNKPQCDGAGGASGLASPGC
SEQ ID NO: 2
Human 25-5′A dependent endoribonuclease/RNase
Lisoform 2 (652 amino acids)
MESRDHNNPQEGPTSSSGRRAAVEDNHLLIKAVQNEDVDLVQQLLEGGAN
VNFQEEEGGWTPLHNAVQMSREDIVELLLRHGADPVLRKKNGATPFILAA
IAGSVKLLKLFLSKGADVNECDFYGFTAFMEAAVYGKVKALKFLYKRGAN
VNLRRKTKEDQERLRKGGATALMDAAEKGHVEVLKILLDEMGADVNACDN
MGRNALIHALLSSDDSDVEAITHLLLDHGADVNVRGERGKTPLILAVEKK
HLGLVQRLLEQEHIEINDTDSDGKTALLLAVELKLKKIAELLCKRGASTD
CGDLVMTARRNYDHSLVKVLLSHGAKEDFHPPAEDWKPQSSHWGAALKDL
HRIYRPMIGKLKFFIDEKYKIADTSEGGIYLGFYEKQEVAVKTFCEGSPR
AQREVSCLQSSRENSHLVTFYGSESHRGHLFVCVTLCEQTLEACLDVHRG
EDVENEEDEFARNVLSSIFKAVQELHLSCGYTHQDLQPQNILIDSKKAAH
LADFDKSIKWAGDPQEVKRDLEDLGRLVLYVVKKGSISFEDLKAQSNEEV
VQLSPDEETKDLIHRLFHPGEHVRDCLSDLLGHPFFWTWESRYRTLRNVG
NESDIKTRKSESEILRLLQPGPSEHSKSFDKWTTKMSKLRHRQIIFPTTQ
NQ

As used herein, the term “sequence identity” means that two nucleotide or amino acid sequences, when optimally aligned, such as by the programs GAP or BESTFIT using default gap weights, share at least, e.g., 70% sequence identity, or at least 80% sequence identity, or at least 85% sequence identity, or at least 90% sequence identity, or at least 95% sequence identity or more. For sequence comparison, typically one sequence acts as a reference sequence (e.g., parent sequence), to which test sequences are compared. The sequence identity comparison can be examined throughout the entire length of a given protein, or within a desired fragment of a given protein. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology). One example of algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (publicly accessible through the National Institutes of Health NCBI internet server). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In certain embodiments, the DNA oligonucleotide molecule also comprises a 2′-O-methyl RNA base at the 5′ end, the 3′ end or both the 5′ and 3′ ends of the DNA oligonucleotide molecule. In certain embodiments, the DNA oligonucleotide molecule comprising 2′-O-methyl RNA base modification in addition to a phosphorothioate linkage induces higher level of RNase L in a cell compared to ssDNA comprising either a phosphorothioate linkage or 2′-O-methyl RNA base modification alone.

In some embodiments, the method is useful for activating RNase L in cancer cells, virus-infected cells, or both.

Methods for introducing oligonucleotides into host cells are well known in the art and include, for example, standard transformation and transfection techniques (e.g., electroporation, chemical transformation). A person of ordinary skill in the field of the invention can readily select an appropriate method for introducing a oligonucleotide into host cells.

### EXEMPLIFICATION

### Example 1

Knock-down of Dicer, a dsRNA processing enzyme involved in micro-RNA biogenesis, has been shown to cause dsRNA accumulation associated with several pathological phenotypes (Kaneko, H. et. al., “Dicer1 deficit induces Alu toxicity in age related macular degeneration,” Nature. 471: (7338): 325-330 (2011), and Donovan, J. et. al., “Rapid RNase L-driven arrest of protein synthesis in the dsRNA response without degradation of translation machinery,” RNA, 23: 1660-1671 (2017)).

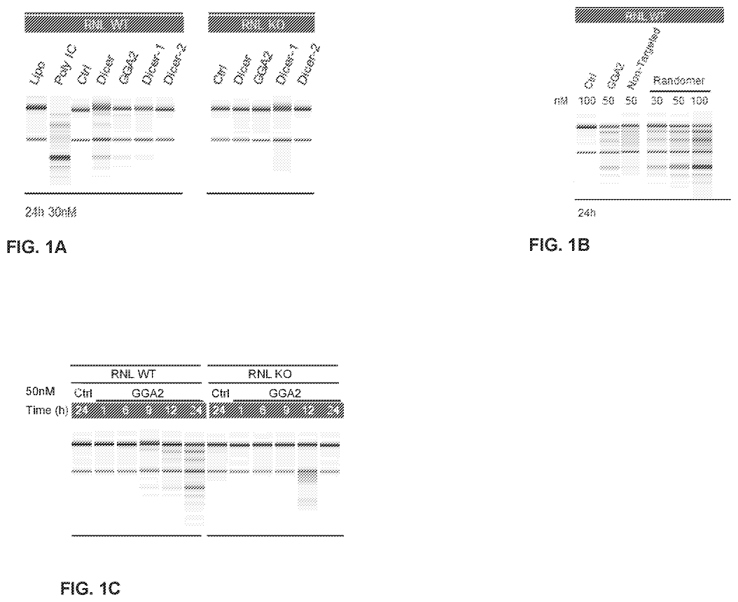

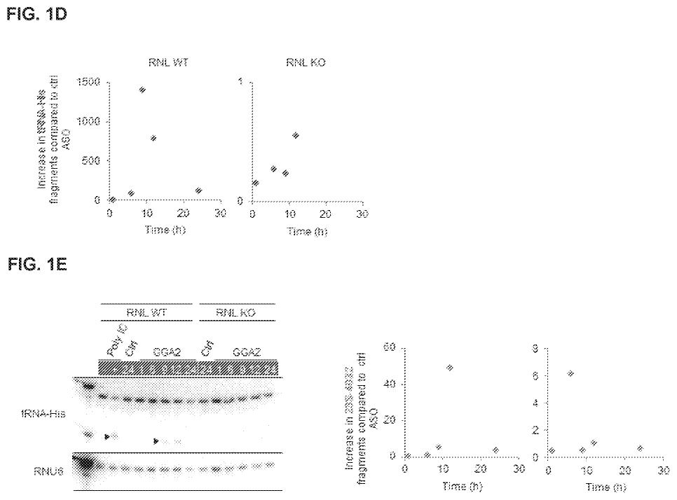

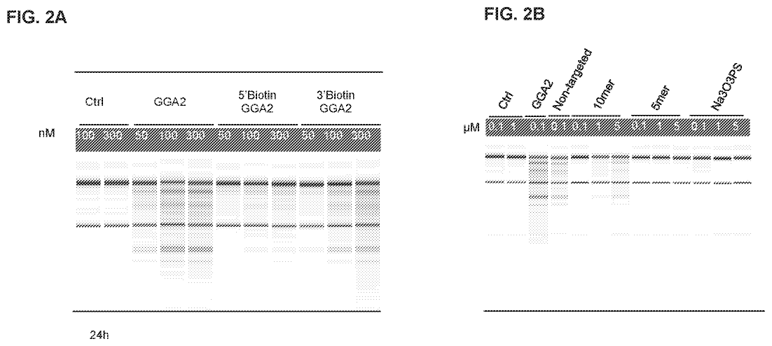

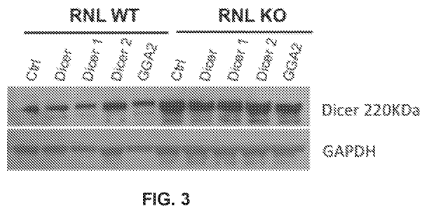

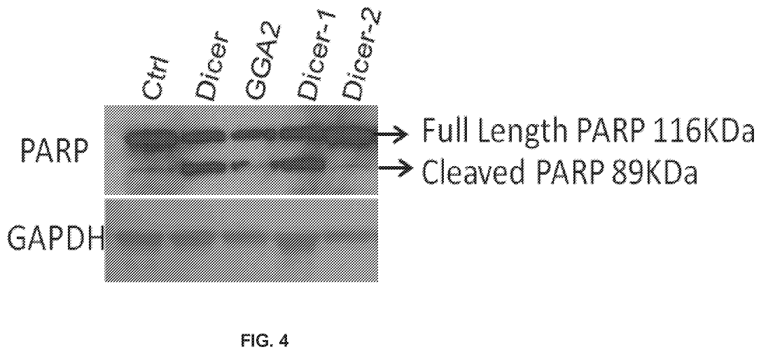

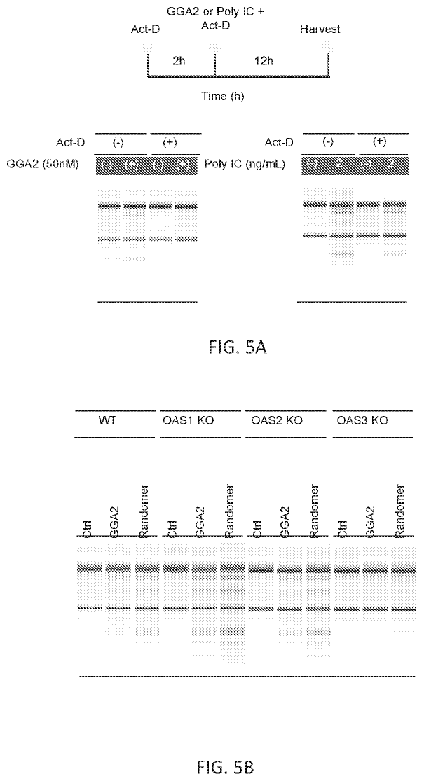

Antisense oligonucleotides were used to test whether knock down of Dicer activates RNase L as a stress response. FIGS. 1-3 collectively show that oligonucleotides with phosphorothioate modifications can activate RNase L independent of their sequence and independent of Dicer protein levels. Along with a phosphorothioate modification, adding 2′ O-methyl RNA bases to the ends of the oligonucleotide sequence was shown to enhance (e.g., synergistically enhance) RNase L activation. FIG. 4 shows that only DNA oligonucleotides containing phosphorothioate modification results in cleavage of full length PARP.

The following materials and methods were used in the experiments described in FIGS. 1-4 herein.

Cell Treatments

For all experiments, A549 cells were plated on 12 well dishes such that they were 80 percent confluent at the time of treatment. Oligonucleotides were transfected into cells using 4 μL Lipofectamine 2000 for 24 hrs. RNA was extracted from cells using the RNeasy kit (Qiagen) and run on a Bioanalyzer. Oligonucleotides used for transfection into cells comprised of anti-sense oligonucleotides directed against Dicer, containing either phosphodiester linkages; or 2′-O-methyl bases at 5′ ad 3′ ends; or both, in addition to other oligonucleotides of varying lengths and phosphodiester linkages; or 2′-O-methyl base compositions as described in Table 1. oligonucleotides with phosphodiester linkages were used as control.

RtcB Quantitative Polymerase Chain Reaction

After treatment with oligonucleotide, total RNA was extracted from cells at the indicated time points using Trizol reagent. RNA was ligated to an adaptor using RtcB and RT-qPCR was carried out to detect accumulation of tRNA-His and 28S-4032 fragments as described previously (8).

Northern Blotting

Trizol purified RNA was resolved on Novex TBE-urea 15 percent polyacrylamide gels (Life Technologies) followed by transfer and UV crosslinking to Brightstar-Plus positively charged nylon membranes (Ambion). Blots were pre-hybridized in Ultrahyb-Oligo (Ambion) followed by hybridization of 5′-³²P-labeled DNA oligonucleotide probes (Probe sequences are specified here²). Membranes were then washed twice with 2×SSC (300 mM NaCl, 30 mM sodium citrate pH 7.0, 0.5 percent SDS) and exposed to phosphor-storage screens. Prior to re-probing, membrane were stripped with 2×10 min washes in near-boiling H₂O/0.5 percent SDS.

Western Blotting

24 hrs after transfection with the oligonucleotide using 4 μL Lipofectamine 2000, cells were lysed in samples buffer (NuPage), separated on 10 percent BisTris PAGE (NuPage) and transferred to PVDF membranes (Life Technologies). Membranes were blocked in 5 percent nonfat dry milk in TBST for 30 mins and probed with 1:1000 rabbit anti-Dicer (Cell Signaling) or 1:1000 rabbit anti-PARP (Cell Signaling) or 1:5000 mouse anti-human GAPDH (Sigma) primary antibodies 4° C. overnight. The membranes were then washed with TBST and incubated with horseradish peroxidase conjugated anti-rabbit or anti-mouse secondary antibodies (1:10,000 Jackson ImmunoResearch) for 30 min. The membranes were washed again and detected with ECL Western Blotting Detection Reagents (GE Healthcare Life Sciences) on an X-ray film.

### Example 2

### Phosphorothioate Oligonucleotide-Mediated Stress Induces Double Stranded RNA

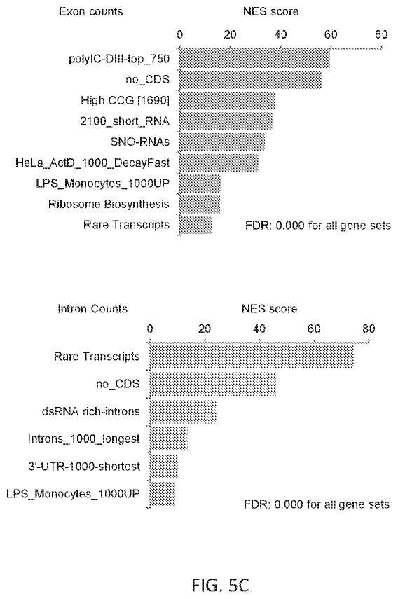

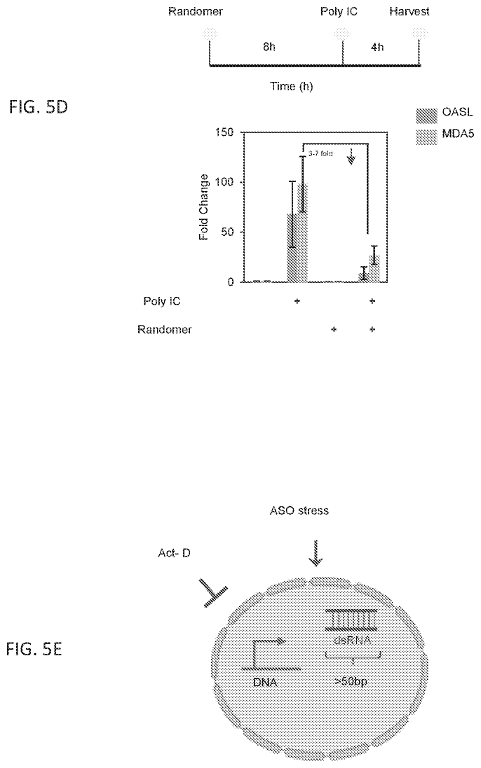

After identifying and characterizing the stressor as a phosphorothioate oligonucleotide, identification of the mechanism by which this modified oligonucleotide activated a dsRNA sensing pathway like OAS-RNase L was explored. Insights into the mechanism of activation were gained when the modified oligonucleotide was tested in the presence of a transcriptional inhibitor like Actinomycin D. Cells pretreated with Actinomycin D did not activated RNase L as shown by the lack of rRNA cleavage upon modified oligonucleotide treatment (FIG. 5A). Actinomycin D does not inhibit any component in the OAS-RNase L signaling pathway because cells treated with Poly IC showed RNase L activation even in the presence of Actinomycin D (FIG. 5A). This suggested that the modified oligonucleotide induced a dsRNA response by active transcription, and Actinomycin D treatment relieved this stress by inhibiting this transcriptional response.

In order to obtain size estimates of the induced dsRNA with the modified oligonucleotide, the modified oligonucleotide was tested in cells with individual OASs knocked out. Among the OASs, OAS3 has a strong preference for dsRNA longer than 50 bp while OAS1 can be activated by dsRNA longer than 18 bp. When individual OASs knocked-out A549 cells were treated with the modified oligonucleotide, it was observed that rRNA cleavage, and hence RNase L activation, was dependent on OAS3 (FIG. 5B). This suggested that the modified oligonucleotide induced dsRNA that was longer than 50 bp.

To identify transcripts induced in this dsRNA stress response, RNA samples were analyzed from modified oligonucleotide treated cells with Ribozero RNA-seq. Fold changes in exon counts were analyzed upon modified oligonucleotide treatment. Using Gene Set Enrichment Analysis, it was found that the modified oligonucleotide induced the same class of inflammatory genes that were induced by Poly IC and LPS (FIG. 5C, top). When intron counts were determined, a significant enrichment for a class of genes which have dsRNA rich introns was observed (FIG. 5C, bottom). These dsRNA rich intron containing genes have repeat elements like Alu and L1 in inverted orientations which can fold to give rise to immunogenic dsRNA.

Cells respond to dsRNA by mediating an interferon response to alert itself and surrounding cells of the presence of this danger signal. As shown in FIG. 5D, Poly IC treated cells show an interferon response as indicated by the up-regulation of signature interferon stimulated genes (ISGs) like OASL and MDA5. However, in cells treated with the modified oligonucleotide, even though it was observed that a transcriptional dsRNA response mediated RNase L activation, an IFN response was not detected, as indicated by the lack of ISG up-regulation with qPCR (FIG. 5D). IFN signatures are absent in our RNA-seq data as well.

Methods

Tissue Culture

Cells were grown using ATCC (American Type Culture Collection) or provider recommended conditions in MEM media+10% FBS (HeLa) or RPMI media+10% FBS (A549), or DMEM media+10% FBS (293T). All media were purchased from Gibco, Life Technologies. HeLa and 293T were a gift from Yibin Kang (Princeton University, Princeton, N.J.). WT and RNase L KO and OAS KOs A549 were a gift from Susan Weiss (University of Pennsylvania, Philadelphia, Pa.). Luminescence assays in live cells were carried out in a plate reader or in 12-well plates at 37° C.

Cell Treatments

For all experiments, cells were plated on 12 well dishes to reach 80 percent confluency at the time of treatment. Oligonucleotides were transfected in cells using 4 μL Lipofectamine 2000 for the specified time periods. RNA was extracted from cells using the RNeasy kit (Qiagen) and run on a Bioanalyzer. For Actinomycin D treatment, cells were pretreated with 1 μg/mL Actinomycin D for 2 hrs. After two hours, media was changed and cells were transfected with the indicated dose of the oligonucleotide using 4 uL Lipofectamine 2000 in fresh media containing 1 μg/mL Actinomycin D. Cells were harvested after 12 hrs in 350 μL RLT buffer.

qRT-PCR Analysis

Cells were harvested in 350 μL RLT buffer (Qiagen) and RNA was purified according to the RNeasy protocol (Qiagen). cDNA was prepared using Random hexamer and a High Capacity RNA to cDNA kit (Applied Biosystems). qPCR was performed using the Power SYBR green PCR mix in a 96 well format on StepOnePlus qPCR instrument (Life Technologies). qPCR primers used in this work are listed in the Table 2 below.

TABLE 2

| Gene | Forward | Reverse |
|---|---|---|
| MDA5 | GCTTCTAGTTAGAGACGTCTTGG | CTTACACCTGATTCATTTCCATT |
| | (SEQ ID NO. 12) | (SEQ ID NO. 13) |
| OASL | GAGCAGAGAGTCCCCGAT | CTGGGAGTTGGGAAGAGAAG |
| | (SEQ ID NO. 14) | (SEQ ID NO. 15) |
| GAPDH | TGTAGTTGAGGTCAATGAAGGG | ACATCGCTCAGACACCATG |
| | (SEQ ID NO. 16) | (SEQ ID NO. 17) |

Ribo-Zero RNA-Seq Experiments

Total RNA was extracted (RNeasy kit, Qiagen). RNA integrity was verified by an RNA 6000 Nano Chip using BioAnalyzer and 2100 Expert software (Agilent Technologies). rRNA was depleted from the total RNA by hybridization to bead bound rRNA probes. This was followed by fragmentation, adapter ligation, PCR amplification and finally sequencing on Illumina HiSEq 2000 platform. Sequencing reads were mapped to the human genome hg19 using TopHat 2 set to map stranded reads with default parameters. Mapped read counts were obtained using HT-seq count run in union mode. The reads were normalized using ACTB1 and GAPDH housekeeping genes and genes with at least 32 reads were included in the analysis.

Unless defined otherwise, all technical and scientific terms used herein have the same meaning as commonly understood by one of ordinary skill in the art to which this invention pertains.

As used herein, the indefinite articles “a” and “an” should be understood to mean “at least one” unless clearly indicated to the contrary.

The phrase “and/or”, as used herein, should be understood to mean “either or both” of the elements so conjoined, i.e., elements that are conjunctively present in some cases and disjunctively present in other cases.

It should also be understood that, unless clearly indicated to the contrary, in any methods described herein that include more than one step or act, the order of the steps or acts of the method is not necessarily limited to the order in which the steps or acts of the method are recited.

Unless otherwise indicated or otherwise evident from the context and understanding of one of ordinary skill in the art, values that are expressed as ranges can assume any specific value or subrange within the stated ranges in various embodiments, unless the context clearly dictates otherwise.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A composition comprising a DNA oligonucleotide molecule, wherein the DNA oligonucleotide molecule comprises:
a) two or more phosphorothioate linkages; and
b) a 2′-O-methyl RNA base at the 5′ end, the 3′ end or both the 5′ and 3′ ends of the oligonucleotide molecule;
wherein the DNA oligonucleotide molecule comprises at least one strand of more than 10 contiguous deoxyribonucleotide bases.

2. The composition of claim 1, wherein the DNA oligonucleotide molecule comprises a deoxyribonucleotide sequence that is not identical to a mammalian genomic deoxyribonucleotide sequence of the same length.

3. The composition of claim 1, wherein the DNA oligonucleotide molecule comprises a deoxynucleotide sequence that has less than 50% identity to a mammalian genomic deoxyribonucleotide sequence of the same length.

4. The composition of claim 1, wherein the DNA oligonucleotide molecule comprises a phosphorothioate linkage between each of the nucleotide bases in at least one of the strands of the DNA oligonucleotide molecule.

5. The composition of claim 1, wherein the DNA oligonucleotide molecule comprises at least one strand of about 23 contiguous deoxyribonucleotide bases.

6. The composition of claim 1, wherein the DNA oligonucleotide molecule is a single stranded DNA (ssDNA) molecule.

7. The composition of claim 1, wherein the DNA oligonucleotide is a double stranded DNA (dsDNA) molecule.

8. The composition of claim 1, wherein the composition comprises a plurality of the DNA oligonucleotide molecules, wherein each DNA oligonucleotide molecule comprises a unique sequence of deoxyribonucleotide bases relative to other DNA oligonucleotide molecules in the plurality.

9. The composition of claim 1, further comprising a pharmaceutically acceptable carrier.

10. The composition of claim 1, further comprising a liposome, a nanoparticle, a micelle or an exosome, wherein the DNA oligonucleotide molecule is contained in the liposome, nanoparticle, micelle or exosome.

11. The composition of claim 1, wherein the concentration of DNA oligonucleotide molecules in the composition is at least about 50 nM.

12. The composition of claim 1, wherein the concentration of the DNA oligonucleotide molecules in the composition is at least about 300 nM.

13. A method for treating cancer in a subject in need thereof, comprising the step of administering to the subject an effective amount of a DNA oligonucleotide molecule, wherein the DNA oligonucleotide molecule comprises two or more phosphorothioate linkages, and wherein the DNA oligonucleotide molecule comprises at least one strand of more than 10 contiguous deoxyribonucleotide bases.

14. The method of claim 13, wherein the DNA oligonucleotide molecule comprises a 2′-O-methyl RNA base at the 5′ end, the 3′ end or both the 5′ and 3′ ends of at least one strand of the DNA oligonucleotide molecule.

15. The method of claim 13, wherein the DNA oligonucleotide molecule comprises a phosphorothioate linkage between each of the nucleotide bases of at least one strand of the DNA oligonucleotide molecule.

16. The method of claim 13, wherein administration of the DNA oligonucleotide molecule inhibits proliferation of cancer cells in the subject.

17. The method of claim 13, wherein administration of the DNA oligonucleotide molecule activates one or more immune system pathways in the subject.

18. The method of claim 17, wherein the immune system pathway is a RNase L pathway.

19. The method of claim 13, further comprising administering one or more additional therapeutic agents to the subject.

20. The method of claim 19, wherein the one or more additional therapeutic agents include a chemotherapeutic agent.

21. The method of claim 19, wherein the one or more additional therapeutic agents include an immunomodulatory agent.

22. The method of claim 13, wherein the DNA oligonucleotide molecule is a single stranded DNA (ssDNA) molecule.

23. The method of claim 13, wherein the DNA oligonucleotide molecule is a double stranded DNA (dsDNA) molecule.

24. A method for activating an RNase L enzyme in a cell, comprising the step of contacting the cell with an effective amount of a DNA oligonucleotide molecule, wherein the DNA oligonucleotide molecule comprises two or more phosphorothioate linkages, and wherein the DNA oligonucleotide molecule comprises at least one strand of more than 10 contiguous deoxyribonucleotide bases.

25. The method of claim 24, wherein the DNA oligonucleotide molecule comprises a 2′-O-methyl RNA base at the 5′ end, the 3′ end or both the 5′ and 3′ ends of at least one strand of the DNA oligonucleotide molecule.

26. The method of claim 24, wherein the cell is a cancer cell.

27. The method of claim 24, wherein the cell is a virus-infected cell.

28. The method of claim 24, wherein the DNA oligonucleotide molecule is a single stranded DNA (ssDNA) molecule.

29. The method of claim 24, wherein the DNA oligonucleotide molecule is a double stranded DNA (dsDNA) molecule.


SEQUENCE LISTING (17 sequences)

SEQ ID NO: 1 - protein, 741 aa, Homo sapiens
MESRDHNNPQEGPTSSSGRRAAVEDNHLLIKAVQNEDVDLVQQLLEGGANVNFQEEEGGWTPLHNAVQMSREDIVELLLRHGADPVLRKKNGATPFILAAIAGSVKLLKLFLSKGADVNECDFYGFTAFMEAAVYGKVKALKFLYKRGANVNLRRKTKEDQERLRKGGATALMDAAEKGHVEVLKILLDEMGADVNACDNMGRNALIHALLSSDDSDVEAITHLLLDHGADVNVRGERGKTPLILAVEKKHLGLVQRLLEQEHIEINDTDSDGKTALLLAVELKLKKIAELLCKRGASTDCGDLVMTARRNYDHSLVKVLLSHGAKEDFHPPAEDWKPQSSHWGAALKDLHRIYRPMIGKLKFFIDEKYKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 2 - protein, 652 aa, Homo sapiens
MESRDHNNPQEGPTSSSGRRAAVEDNHLLIKAVQNEDVDLVQQLLEGGANVNFQEEEGGWTPLHNAVQMSREDIVELLLRHGADPVLRKKNGATPFILAAIAGSVKLLKLFLSKGADVNECDFYGFTAFMEAAVYGKVKALKFLYKRGANVNLRRKTKEDQERLRKGGATALMDAAEKGHVEVLKILLDEMGADVNACDNMGRNALIHALLSSDDSDVEAITHLLLDHGADVNVRGERGKTPLILAVEKKHLGLVQRLLEQEHIEINDTDSDGKTALLLAVELKLKKIAELLCKRGASTDCGDLVMTARRNYDHSLVKVLLSHGAKEDFHPPAEDWKPQSSHWGAALKDLHRIYRPMIGKLKFFIDEKYKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKMSKLRHRQIIFPTTQNQ

SEQ ID NO: 3 - DNA, 19 bp, ARTIFICIAL SEQUENCE
  ANTISENSE OLIGONUCLEOTIDE
gccagatatacgcgttgac

SEQ ID NO: 4 - DNA, 28 bp, ARTIFICIAL SEQUENCE
  ANTISENSE OLIGONUCLEOTIDE
gmcmumamcctttttgctumcmumcmam

SEQ ID NO: 5 - DNA, 33 bp, ARTIFICIAL SEQUENCE
  ANTISENSE OLIGONUCLEOTIDE
cmamumcmumccagcaccgttaagmgmcmamum

SEQ ID NO: 6 - DNA, 20 bp, ARTIFICIAL SEQUENCE
  ANTISENSE OLIGONUCLEOTIDE
gctgacctttttgcttctca

SEQ ID NO: 7 - DNA, 30 bp, ARTIFICIAL SEQUENCE
  ANTISENSE OLIGONUCLEOTIDE
gmcmumgmamcctttttgctumcmumcmam

SEQ ID NO: 8 - DNA, 32 bp, ARTIFICIAL SEQUENCE
  ANTISENSE OLIGONUCLEOTIDE
acactctttccctacacgacgctcttccgatc

SEQ ID NO: 9 - DNA, 23 bp, ARTIFICIAL SEQUENCE
  ANTISENSE OLIGONUCLEOTIDE
  misc_feature (1)..(5): n is gm, cm, um, or am
  misc_feature (6)..(18): n is a, c, g, or t
  misc_feature (19)..(23): n is gm, cm, um, or am
nnnnnnnnnnnnnnnnnnnnnnn

SEQ ID NO: 10 - DNA, 10 bp, ARTIFICIAL SEQUENCE
  ANTISENSE OLIGONUCLEOTIDE
  misc_feature (1)..(10): n is a, c, g, or t
nnnnnnnnnn

SEQ ID NO: 11 - DNA, 5 bp, ARTIFICIAL SEQUENCE
  ANTISENSE OLIGONUCLEOTIDE
  misc_feature (1)..(5): n is a, c, g, or t
nnnnn

SEQ ID NO: 12 - DNA, 23 bp, ARTIFICIAL SEQUENCE
  MDA5 FORWARD PRIMER
gcttctagttagagacgtcttgg

SEQ ID NO: 13 - DNA, 23 bp, ARTIFICIAL SEQUENCE
  MDA5 REVERSE PRIMER
cttacacctgattcatttccatt

SEQ ID NO: 14 - DNA, 18 bp, ARTIFICIAL SEQUENCE
  OASL FORWARD PRIMER
gagcagagagtccccgat

SEQ ID NO: 15 - DNA, 20 bp, ARTIFICIAL SEQUENCE
  OASL REVERSE PRIMER
ctgggagttgggaagagaag

SEQ ID NO: 16 - DNA, 22 bp, ARTIFICIAL SEQUENCE
  GAPDH FORWARD PRIMER
tgtagttgaggtcaatgaaggg

SEQ ID NO: 17 - DNA, 19 bp, ARTIFICIAL SEQUENCE
  GAPDH REVERSE PRIMER
acatcgctcagacaccatg